(19) United States
(12) Patent Application Publication
(10) Pub. No.: US 2021/0106452 A1
(43) Pub. Date: Apr. 15, 2021

(54) Knee Distractor Apparatus and Method

(71) Applicant: Monty L. Ruetenik, Clear Lake Shores, TX (US)

(72) Inventor: Monty L. Ruetenik, Clear Lake Shores, TX (US)

(21) Appl. No.: 16/919,810

(22) Filed: Jul. 2, 2020

Related U.S. Application Data
(63) Continuation-in-part of application No. 15/727,738, filed on Oct. 9, 2017, now abandoned.

(51) Int. Cl. A61F 5/058; A61F 5/042; A61F 5/01

(52) U.S. Cl. CPC A61F 5/05816; A61F 5/042; A61F 5/012; A61F 5/0106; A61F 5/0111

(57) ABSTRACT

An apparatus and method for distraction (stretching and/or straightening) of injured or repaired knee joints. The apparatus comprises pneumatic or hydraulic cylinders designed to be disposed on either side of a leg with a knee to be treated to stretch the leg joint when the cylinders are activated.

### CROSS-REFERENCE TO RELATED APPLICATION

This application is a continuation in part of U.S patent application Ser. No. 15/727,738 filed Oct. 9, 2017 and claims the benefit and priority from U. S. Provisional Patent Application 62/407,783, filed Oct. 13, 2016, the contents and disclosure of both are incorporated herein by reference for all purposes.

### BACKGROUND

### Field

Apparatus and method for distraction (stretching and/or straightening) of injured knee joints.

### Background

After knee surgery and in rehabilitation of sore and injured knees, it is important to distract (stretch or uncouple) the knee joint to prevent stiffness from becoming permanent. In athletes, this is frequently accomplished by having a trainer pull on the leg to distract the knee joint for a predetermined time period−10 to 20 minutes is customary. In some rehabilitation and physical therapy situations, bungee cords are attached to the ankle or a leg that has an injured knee, while the patient lies down with the cord under tension—stretching or uncoupling the knee joint. Bungee cords of varying elasticities allow customization of the tension applied. These methods, however, either require manual assistance or a controlled environment (therapy session) to be satisfactorily applied. A detailed description of the problems in knee rehabilitation and the need for stretching is described in published application U.S. 2014/0088466, together with a complicated apparatus for use in therapy, the disclosure of which is incorporated herein by reference.

### DESCRIPTION OF THE FIGURES

FIG. 1A is a front drawing of an embodiment of the invention showing the knee distractor in position on a leg.

FIG. 1B is a front drawing of another embodiment of the invention showing the knee distractor in position on a leg.

FIG. 1C is a front drawing of another embodiment of the invention showing details of the connection between the knee and cylinder tube.

FIG. 2 is a front drawing of an embodiment of the invention showing the knee distractor components in position.

FIG. 3A is a perspective view of an ankle brace suitable for an embodiment of the invention.

FIG. 3B is another perspective view of an ankle brace suitable for an embodiment of the invention.

FIG. 4 is a perspective view of a knee brace suitable for of an embodiment of the invention.

FIG. 5 is graphical plot showing relationship between displacement and applied pressure of a cylinder of an embodiment of the invention.

### DETAILED DESCRIPTION

In broad aspect, the invention is an apparatus and method for distraction (stretching and/or straightening) of injured or repaired knee joints. The apparatus comprises pneumatic or hydraulic cylinders that are designed to be disposed on either side of a leg with a knee to be treated. It is important that a cylinder be placed on each side of the leg to prevent equalize the pull on the knee joint. If only on one side it is likely that the knee would be pulled sidewise as well as downward. Cylinders are attached to a knee brace or other leg-holding device, secured above the knee joint, and attached to another device secured around the ankle of the leg. The leg-holding device and ankle device are not connected nor hinged together. When the cylinder is activated, (piston extended) the knee brace and ankle device are separated (moved apart) thus causing the knee joint to be straightened or slightly separated.

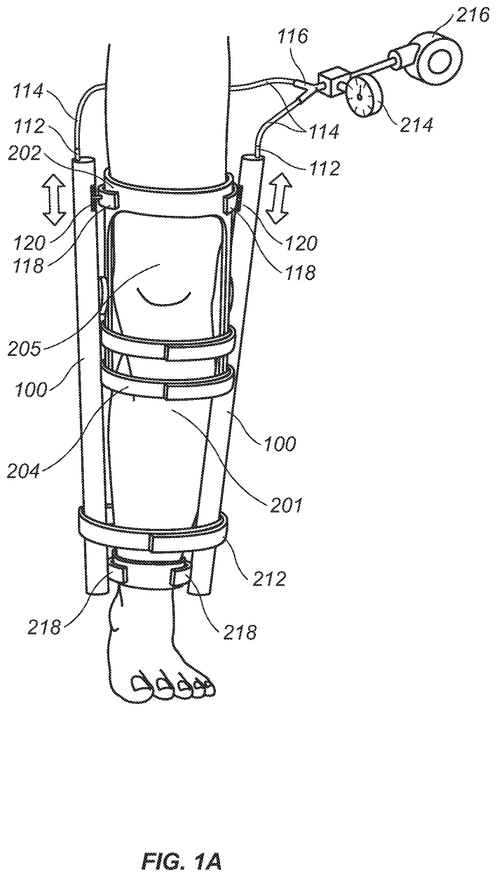

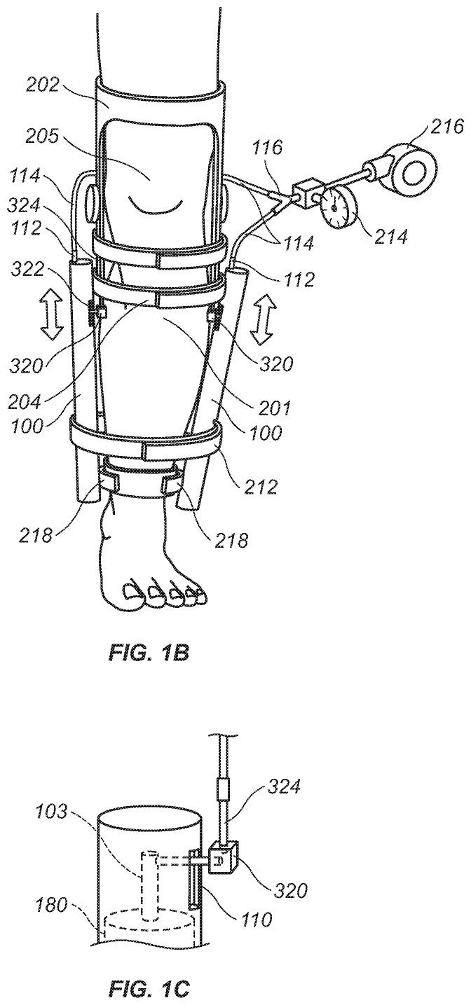

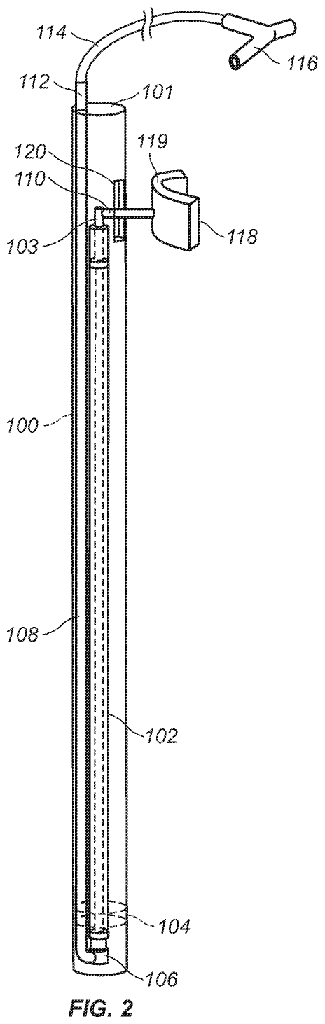

Referring to the drawings, FIG. 1A illustrates a leg 201 with a knee 205 and upper knee brace 202 and lower knee brace 204. Knee stretch cylinders 100 are attached on each side of a leg where connectors 118 are fitted with side straps on the braces 202/204. In use, the lower section of the brace 204 is loosened to allow some small movement upward, and the upper section is secured. The lower section of the tube is removably attached to an ankle brace 212 by a suitable connector, 218, (much like connector 118) and does not move during use. FIG. 1B and 1C illustrate another embodiment of the system. In this embodiment, the piston extension 110 connects to a connection member 320 and 322 that has a slot (or other connection means) to receive a side brace of a flexion (means to set knee bend positions). It is not required that the connection 320 be able to permanently attach to brace side member 324 through 322 since the action is always upward. Rather, it is only necessary that the connection allow 110 to push brace member 324 upward in cylinder 180. This embodiment allows the brace to be set for limited movement of the knee (as often required—as to keep the knee joint from fully straightening) and be pushed upward with the knee in the limited (as for example, slightly bent) position to, for example, separate the joint members without straightening the knee joint. Patents describing flexion set means include U.S. Pat. No. 5,672,152, U.S. Pat. No. 5,827,208, and U.S. Pat. No. 7235059, the relevant disclosures of which are incorporated herein by reference. Applicant is not aware of any known current apparatus that will allow this action of extension of a bent knee joint. Conduits (such as tubing) 114 extend from the knee stretch cylinder 100 (also shown as 101) (see FIG. 2) to a Y connector 116 that is connected to the cylinder at 106 and to air pressure pump 216. Member 214 is a pressure gauge preferably a digital gauge. The gauge allows for consistent, staged, and repeated use of the device. In use in a therapy protocol, the gauge will allow return to (or alteration) of a previous treatment stretch range. FIG. 2 shows details of the knee stretch cylinder-100. The cylinder 100 comprises a straight, unitary, unhinged, hollow tube (such as a carbon or metal tube) 108 in which is disposed a straight unhinged unitary mechanical pneumatic cylinder 102 with an air connector 112 at the bottom and air hose 114 extending from the air hose connector 112 up through the open top of the tube. It is important the hollow tube and cylinder be configured so as not to bend in use so that the extension of the leg will be properly accomplished. The pneumatic cylinder is attached to the lower section of the tube at point 104. It is preferred that the pneumatic cylinder be spring-loaded so that when no air pressure is applied, it will return to its starting position. The pneumatic (or hydraulic) cylinder has a piston 103 and a horizontally (at right angles to the length of the cylinder) arranged rod 110 that extends through a slot 120 in the tube 108. The rod is connected to a brace connector 118, a semicircular member that has a slot 119 into which fits a side arm of a knee brace. When the pneumatic cylinder is activated, the piston moves upward causing the rod and therefore the connector 118 to move upward putting tension between the upper brace and ankle brace, thus stretching the knee joint. In general, the slot 120 and therefore the travel of the rod upward will be about 1 to 4 inches, with 1 to-2 inches preferred. The air hoses 114 that feed the pneumatic cylinder are connected to flexible conduits 114 that are connected by a Y connector 116 to air pump 216.

The upper knee brace is secure so that when the piston is extended, the space between the ankle brace 212 and the upper knee brace 202 is likewise extended putting tension on the knee joint and thereby stretching it. This accomplishes the desirable uncoupling or straightening of the joint—if the knee is bent, the action will tend to straighten it, and if the knee is straight, the action will uncouple or distract the joint.

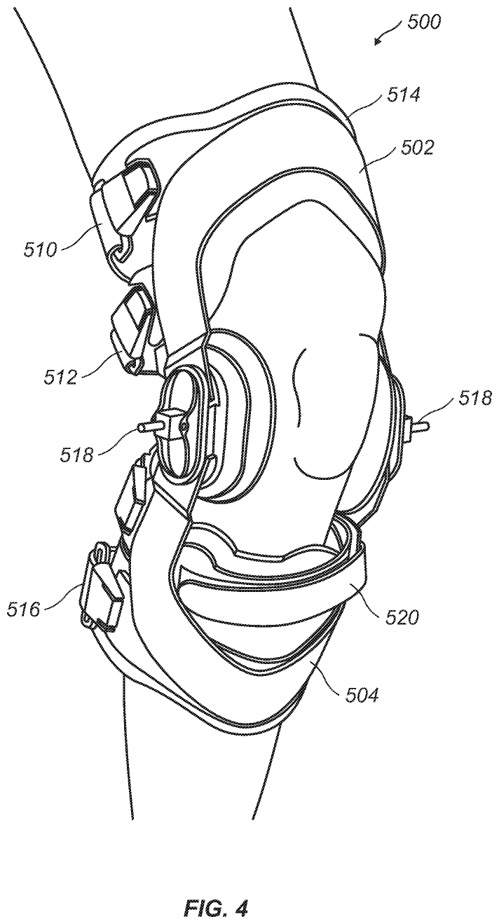

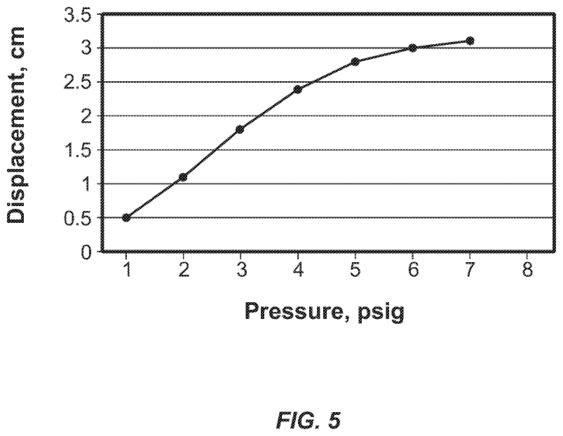

FIG. 4 is an illustration of a suitable knee brace. There is a connection (118 to 518 on each side of the brace) attaching the cylinder tube (as 100 in FIG. 1) to the vertical side straps, 502, of the brace, which generally are only required to hold the strap in upward movement. Additionally, there are leg wrap bands 514 that can be latched via 510, 512 and 516 to tighten the brace 500 around the leg above and below the knee 205. Hook and loop straps 520 and 504 are also used to secure the brace to the leg. Reaction braces from DonJoy™ can be adapted to be very suitable (www.donjoyperformance.com/webtech-knee-brace). In use, the knee brace will be loosened below the knee 205 to allow the more secure top section above the knee to be pushed upward by the cylinder action, thus extending the distance between the knee and ankle and ultimately distracting the knee joint.

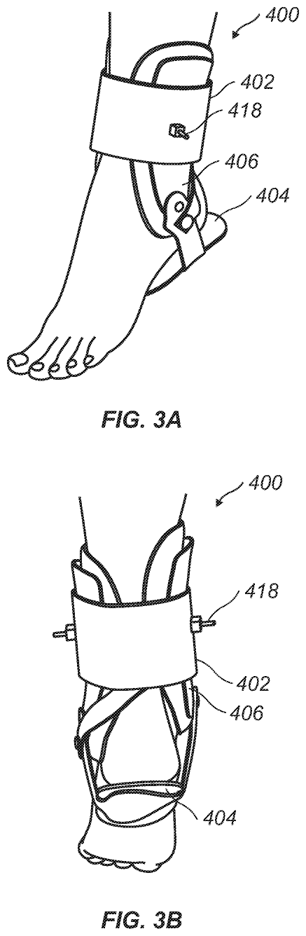

FIG. 3A and 3B illustrate a suitable ankle brace, 400. As with the knee braces, there will be a connection (218 to 418 on each side of the brace) attaching the cylinder tube (as 100 in FIG. 1) to the sides of the brace. This ankle brace has a band 402 and side pads 406 and an underfoot pad 404. Generally, it is only required to hold the brace in position to allow the ankle to be forced downward when the cylinder 100 is extended. In most ankle braces, it is preferred that the foot be placed in a shoe with the brace disposed inside the shoe, in order to ensure the connection to the ankle is secure. An ankle cuff can also be used in the same way as an ankle brace. Suitable ankle braces include T2 Multi-Sport Ankle Brace from Active Ankle™. (see www.activeankle.com)

Suitable cylinders include those available from Automation Direct (cylinders a07020sp—9/16 bore or a07020sp—7/16 bore), preferably those with a spring return.

Suitable hand air pumps include Silverline elite hand pump model 3mitmv8510 available from ToolTopia at tooltopia .com.

### Method of Use

Generally, the apparatus will be fitted by an orthotic surgeon, physical therapist, or athletic/fitness trainer. The apparatus assembly will be fitted and attached to the knee and ankle braces. When properly adjusted, the assembly is tested and characteristics plotted by measuring the extension for a given pressure applied. The air pump (or hydraulic, if used) will be applied at various pressures—e.g. 2, 5, 8, 10 psi, and the displacement (and stretch) of the piston travel (distance from knee brace attachment to ankle brace attachment) measured.

Characteristic Chart (Illustrative)

| Pressure, psi | Displacement, cm |
|---|---|
| 2 | 0.5 |
| 4 | 1.1 |
| 6 | 1.8 |
| 8 | 2.4 |
| 10 | 2.8 |
| 12 | 3.0 |
| 14 | 3.1 |

A characteristic curve such as illustrated in FIG. 5 can be generated for each apparatus/used combination. Using the characteristic curve, a set of user protocol can be prescribed. The user can then use the assembly at any convenient place and time without needing to visit a therapy facility.

If a knee-lock brace is used (one that locks into a predetermined position), the practitioner, physical therapist, or athletic/fitness trainer will specify (and/or set) the brace knee position lock means in the desired treatment position of the knee joint.

In the foregoing specification, the invention has been described with reference to specific embodiments thereof. It will, however, be evident that various modifications and changes can be made thereto without departing from the broader spirit and scope of the invention as set forth in the appended claims. The specification and drawings are, accordingly, to be regarded in an illustrative rather than a restrictive sense. Therefore, the scope of the invention should be limited only by the appended claims.

1. A system for the distraction of human knee joints for stretching and straightening comprising;
two pneumatic or hydraulic cylinders each disposed inside a single hollow tube and one each disposed on each side of a human leg wherein each hollow tube at the proximal end is attached to a knee brace or other leg holding device and at the distal end is attached to an ankle brace or cuff, wherein the knee brace or other leg holding device and ankle brace or cuff are not connected together and wherein there is a pneumatic system adapted to activate the cylinders to move inside the hollow tube and wherein the hollow tube and the pneumatic or hydraulic cylinders are not jointed or hinged and extend from the knee brace to the ankle brace as a unitary structure.

2. The system of claim 1 comprising four cylinders, two cylinders each configured to be placed on each side of each leg of a patient.

3. The system of claim 1 wherein the knee brace is a resistance flexion knee brace.

4. The system of claim 1 wherein the cylinders are pneumatic and means to activate is a hand-operated air pump.

5. The system of claim 1 wherein the top of the cylinder has an extended structure disposed at substantially right angles to the cylinder length that has an end that extends through a slot in the hollow tube.

6. The system of claim 5 wherein the extended structure disposed at right angles to the cylinder length and slot in the hollow tube are configured to limit the movement of a human knee joint.

7. The system of claim 1 wherein the ankle brace has straps to secure the brace around the leg above the ankle.

8. A method of distraction of knee joints comprising fitting a leg with a knee joint with an apparatus comprising two pneumatic or hydraulic cylinders each disposed inside a single hollow tube and one each disposed on each side of a human leg, wherein each hollow tube at the proximal end is attached to a knee brace or other leg holding device and at the distal end is attached to an ankle brace or cuff and wherein there is a pneumatic system adapted to activate the cylinders to move inside the hollow tube and wherein the hollow tube and the pneumatic or hydraulic cylinders are not jointed or hinged and extend from the knee brace to the ankle brace as a unitary structure.

9. The method of claim 8 wherein the knee brace is a flexion brace that has a stop structure to limit the movement of the knee joint to which it is attached.

10. The method of claim 8 wherein the top of the cylinder has an extended structure disposed at substantially right angles to the cylinder length that has an end that extends through a slot in the hollow tube.

11. The method of claim 8 wherein the extended structure disposed at right angles to the cylinder length and slot in the hollow tube are configured to limit the movement of a human knee joint.

12. The method of claim 8 comprising constructing a data array or chart correlating air or hydraulic pressure with piston extension and using the chart to construct a protocol for treatment distraction of knee joints.
